SEQUENCE LISTING (11 sequences)

SEQ ID NO: 1 - protein, 588 aa, Arabidopsis sp.
MAMVDEPLYPIAVLIDELKNDDIQLRLNSIRRLSTIARALGEERTRKELIPFLSENSDDDDEVLLAMAEELGVFIPFVGGIEFAHVLLPPLESLCTVEETCVREKAVESLCKIGSQMKENDLVESFVPLVKRLAGGEWFAARVSACGIFHVAYQGCTDVLKTELRATYSQLCKDDMPMVRRAAASNLGKFATTVESTFLIAEIMTMFDDLTKDDQDSVRLLAVEGCAALGKLLEPQDCVARILPVIVNFSQDKSWRVRYMVANQLYELCEAVGPDCTRTDLVPAYVRLLRDNEAEVRIAAAGKVTKFCRLLNPELAIQHILPCVKELSSDSSQHVRSALASVIMGMAPILGKDSTIEHLLPIFLSLLKDEFPDVRLNIISKLDQVNQVIGIDLLSQSLLPAIVELAEDRHWRVRLAIIEYVPLLASQLGIGFFDDKLGALCMQWLQDKVYSIREAAANNLKRLAEEFGPEWAMQHLVPQVLDMVNNPHYLHRMMVLRAISLMAPVMGSEITCSKFLPVVVEASKDRVPNIKFNVAKLLQSLIPIVDQSVVDKTIRQCLVDLSEDPDVDVRYFANQALNSIDGSTAAQS

SEQ ID NO: 2 - protein, 587 aa, Arabidopsis sp.
MSMIDEPLYPIAVLIDELKNDDIQLRLNSIRRLSTIARALGEERTRKELIPFLSENNDDDDEVLLAMAEELGVFIPYVGGVEYAHVLLPPLETLSTVEETCVREKAVESLCRVGSQMRESDLVDHFISLVKRLAAGEWFTARVSACGVFHIAYPSAPDMLKTELRSLYTQLCQDDMPMVRRAAATNLGKFAATVESAHLKTDVMSMFEDLTQDDQDSVRLLAVEGCAALGKLLEPQDCVQHILPVIVNFSQDKSWRVRYMVANQLYELCEAVGPEPTRTELVPAYVRLLRDNEAEVRIAAAGKVTKFCRILNPEIAIQHILPCVKELSSDSSQHVRSALASVIMGMAPVLGKDATIEHLLPIFLSLLKDEFPDVRLNIISKLDQVNQVIGIDLLSQSLLPAIVELAEDRHWRVRLAIIEYIPLLASQLGVGFFDDKLGALCMQWLQDKVHSIRDAAANNLKRLAEEFGPEWAMQHIVPQVLEMVNNPHYLYRMTILRAVSLLAPVMGSEITCSKLLPVVMTASKDRVPNIKFNVAKVLQSLIPIVDQSVVEKTIRPGLVELSEDPDVDVRFFANQALQSIDNVMMSS

SEQ ID NO: 3 - protein, 587 aa, Arabidopsis sp.
MSMVDEPLYPIAVLIDELKNDDIQRRLNSIKRLSIIARALGEERTRKELIPFLSENNDDDDEVLLAMAEELGGFILYVGGVEYAYVLLPPLETLSTVEETCVREKAVDSLCRIGAQMRESDLVEHFTPLAKRLSAGEWFTARVSACGIFHIAYPSAPDVLKTELRSIYGQLCQDDMPMVRRAAATNLGKFAATIESAHLKTDIMSMFEDLTQDDQDSVRLLAVEGCAALGKLLEPQDCVAHILPVIVNFSQDKSWRVRYMVANQLYELCEAVGPEPTRTDLVPAYARLLCDNEAEVRIAAAGKVTKFCRILNPELAIQHILPCVKELSSDSSQHVRSALASVIMGMAPVLGKDATIEHLLPIFLSLLKDEFPDVRLNIISKLDQVNQVIGIDLLSQSLLPAIVELAEDRHWRVRLAIIEYIPLLASQLGVGFFDEKLGALCMQWLQDKVHSIREAAANNLKRLAEEFGPEWAMQHIVPQVLEMINNPHYLYRMTILRAVSLLAPVMGSEITCSKLLPAVITASKDRVPNIKFNVAKMMQSLIPIVDQAVVENMIRPCLVELSEDPDVDVRYFANQALQSIDNVMMSS

SEQ ID NO: 4 - protein, 585 aa, Glycine max
MADEPLYPIAVLIDELKNDDIQLRLNSIRRLSTIARALGEERTRRELIPFLSENNDDDDEVLLAMAEELGVFIPYVGGVEHASVLLPPLETLCTVEETCVRDKAVESLCRIGSQMRESDLVEYYIPLVKRLAAGEWFTARVSACGLFHIAYPSAPETSKTELRSIYSQLCQDDMPMVRRSAASNLGKFAATVEYAHLKADVMSIFDDLTQDDQDSVRLLAVEGCAALGKLLEPQDCVAHILPVIVNFSQDKSWRVRYMVANQLYELCEAVGPEPTRTELVPAYVRLLRDNEAEVRIAAAGKVTKFCRILNPDLAIQHILPCVKELSSDSSQHVRSALASVIMGMAPVLGKEATIEQLLPIFLSLLKDEFPDVRLNIISKLDQVNQVIGIDLLSQSLLPAIVELAEDRHWRVRLAIIEYIPLLASQLGVRFFDDKLGALCMQWLQDKVHSIREAAANNLKRLAEEFGPEWAMQHIIPQVLEMNNNPHYLYRMTILRAISLLAPVMGPEITCSNLLPVVLAASKDRVPNIKFNVAKVLESIFPIVDQSVVEKTIRPCLVELSEDPDVDVRFFSNQALQAIDHVMMSC

SEQ ID NO: 5 - protein, 585 aa, Glycine max
MADEPLYPIAVLIDELKNDDIQLRLNSIRRLSTIARALGEERTRRELIPFLSENNDDDDEVLLAMAEELGVFIPYVGGVEHASVLLPPLETLCTVEETCVRDKAAESLCRIGSQMRESDLVEYYIPLVKRLAAGEWFTARVSACGLFHIAYPSAPETSKTELRSIYSQLCQDDMPMVRRSAASNLGKFAATVEYAHLKADLMSIFDDLTQDDQDSVRLLAVEGCAALGKLLEPQDCVAHILPVIVNFSQDKSWRVRYMVANQLYELCEAVGPEPTRTELVPAYVRLLRDNEAEVRIAAAGKVTKFCRILNPDLSIQHILSCVKELSSDSSQHVRSALASVIMGMAPVLGKEATIEQLLPIFLSLLKDEFPDVRLNIISKLDQVNQVIGIDLLSQSLLPAIVELAEDRHWRVRLAIIEYIPLLASQLGVSFFDDKLGALCMQWLQDKVHSIREAAANNLKRLAEEFGPEWAMQHIIPQVLEMNNNPHYLYRMTILRAISLLAPVMGPEITCSNLLPVVVAASKDRVPNIKFNVAKVLESIFPIVDQSVVEKTIRPCLVELSEDPDVDVRFFSNQALQAIDHVMMSS

SEQ ID NO: 6 - protein, 587 aa, Glycine max
MAMVDQPLYPIAVLIDELKNEDIQLRLNSIRRLSTIARALGEDRTRKELIPFLSENNDDDDEVLLAMAEELGVFIPYVGGVDHANVLLPPLETLCTVEETCVRDKSVESLCRIGAQMREQDLVEHFIPLVKRLAAGEWFTARVSSCGLFHIAYPSAPESVKTELRAIYGQLCQDDMPMVRRSAATNLGKFAATVEAPHLKSDIMSVFEDLTQDDQDSVRLLAVEGCAALGKLLEPQDCVAHILPVIVNFSQDKSWRVRYMVANQLYELCEAVGPDPTRSELVPAYVRLLRDNEAEVRIAAAGKVTKFSRILNPDLAIQHILPCVKELSTDSSQHVRSALASVIMGMAPVLGKDATIEQLLPIFLSLLKDEFPDVRLNIISKLDQVNQVIGIDLLSQSLLPAIVELAEDRHWRVRLAIIEYIPLLASQLGVGFFDDKLGALCMQWLKDKVYSIRDAAANNIKRLAEEFGPDWAMQHIIPQVLDMVTDPHYLYRMTILQAISLLAPVLGSEITSSKLLPLVINASKDRVPNIKFNVAKVLQSLIPIVDQSVVESTIRPCLVELSEDPDVDVRFFASQALQSSDQVKMSS

SEQ ID NO: 7 - protein, 587 aa, Glycine max
MSMVDEPLYPIAVLIDELKNDDIQLRLNSIRKLSTIARALGEERTRRELIPFLGENNDDDDEVLLAMAEELGVFIPFVGGVEHAHVLLPPLEMLCTVEETCVRDKAVESLCRIGLQMRESDLVEYFIPLVKRLASGEWFTARVSSCGLFHIAYPSAPEMSKIELRSMYSLLCQDDMPMVRRSAASNLGKYAATVEYAHLKADTMSIFEDLTKDDQDSVRLLAVEGCAALGKLLEPQDCITHILPVIVNFSQDKSWRVRYMVANQLYELCEAVGPEPTRTELVPAYVRLLRDNEAEVRIAAAGKVTKFCRILNPDLSIQHILPCVKELSTDSLQHVRSALASVIMGMAPVLGKDATIEQLLPIFLSLLKDEFPDVRLNIISKLDQVNQVIGINLLSQSLLPAIVELAEDRHWRVRLAIIEYIPLLASQLGVGFFYDKLGALCMQWLQDKVHSIREAAANNLKRLAEEFGPEWAMQHIIPQVLEMISNPHYLYRMTILHAISLLAPVMGSEITRSELLPIVITASKDRVPNIKFNVAKVLESIFPIVDQSVVEKTIRPSLVELSEDPDVDVRFFSNQALHAMDHVMMSS

SEQ ID NO: 8 - protein, 587 aa, Glycine max
MAMVDQPLYPIAVLIDELKNEDIQLRLNSIRRLSTIARALGEDRTRKELIPFLSENNDDDDEVLLAMAEELGVFIPYVGGVEHANVLLPPLETLCTVEETSVRDKSVESLCRIGAQMREQDLVEYLIPLVKRLAAGEWFTARVSSCGLFHIAYPSAPEAVKTELRAIYGQLCQDDMPMVRRSAATNLGKFAATVEAPHLKSDIMSVFEDLTHDDQDSVRLLAVEGCAALGKLLEPQDCVAHILPVIVNFSQDKSWRVRYMVANQLYELCEAVGPDPTRSELVPAYVRLLRDNEAEVRIAAAGKVTKFSRILNPDLAIQHILPCVKELSTDSSQHVRSALASVIMGMAPVLGKDATIEQLLPIFLSLLKDEFPDVRLNIISKLDQVNQVIGIDLLSQSLLPAIVELAEDRHWRVRLAIIEYIPLLASQLGVSFFDDKLGALCMQWLKDKVYSIRDAAANNIKRLAEEFGPDWAMQHIIPQVLDMVTDPHYLYRMTILQSISLLAPVLGSEISSSKLLPLVINASKDRVPNIKFNVAKVLQSLIPIVDQSVVESTIRPCLVELSEDPDVDVRFFASQALQSCDQVKMSS

SEQ ID NO: 9 - protein, 521 aa, Solanum lycopersicum
MAEELGVFIPYVGGVEHAHVLLPPLETLCTVEETCVRDKAVESLCRIGSQMRESDLVDWFVPLVKRLAAGEWFTARVSACGLFHIAYSSAPEMLKAELRSIYSQLCQDDMPMVRRSAATNLGKFAATVESAYLKSDIMSIFDDLTQDDQDSVRLLAVEGCAALGKLLEPQDCVAHILPVIVNFSQDKSWRVRYMVANQLYELCEAVGPEPTRTDLVPAYVRLLRDNEAEVRIAAAGKVTKFCRILSPELAIQHILPCVKELSSDSSQHVRSALASVIMGMAPVLGKDATIEHLLPIFLSLLKDEFPDVRLNIISKLDQVNQVIGIDLLSQSLLPAIVELAEDRHWRVRLAIIEYIPLLASQLGIGFFDDKLGALCMQWLQDKVYSIRDAAANNLKRLAEEFGPEWAMQHIIPQVLDMTTSPHYLYRMTILRSISLLAPVMGSEITCSKLLPVVVTATKDRVPNIKFNVAKVLQSLVPIVDNSVVEKTIRPSLVELAEDPDVDVRFYANQALQSIDNVMMSG

SEQ ID NO: 10 - protein, 581 aa, Solanum lycopersicum
MSAIDEPLYPIAVLIDELKNEDIQLRLNSIRRLSTIARALGEERTRKELIPFLSENNDDDDEVLLAMAEELGMFIPYVGGVEHARVLLPPLEGLCSVEETCVREKAVESLCKIGSQMKESDLVESFIPLVKRLATGEWFTARVSSCGLFHIAYPSAPEPLKNELRTIYSQLCQDDMPMVRRAAATNLGKFAATIEQPHLKTDIMSMFETLTQDDQDSVRLLAVEDCAALGKLLEPKDCVAQILSVIVNFAQDKSWRVRYMVANQLYDLCEAVGPEATRTDLVPAYVRLLRDNEAEVRIAAAGKVTKFCRILSPELAIQHILPCVKELSSDSSQHVRSALASVIMGMAPILGKDATIEQLLPIFLSLLKDEFPDVRLNIISKLDQVNQVIGIDLLSQSLLPAIVELAEDRHWRVRLAIIEYIPLLASQLGVGFFDDKLGALCMQWLKDKVYSIRDAAANNVKRLAEEFGPKWAMEHIIPQVLDMINDPHYLYRMTILHAISLLAPVLGSEIACSKLLPVIITASKDRVPNIKFNVAKVLQSVIPIVEQSVVESTIRPCLVELSEDPDVDVRFFANQALQATK

SEQ ID NO: 11 - protein, 587 aa, Solanum lycopersicum
NSCTLSKPFDHFCLLSPNTFHFIEINEGNKSSLLNSPDIKGFTSPAAGDTHFRCKGNTIYLSMAHLLLYPMILDELKNDDIQLRLNSVRRLSSIACQLGEDRTRRELIPFLCRNTDDEDEVLLAMSEELGGFIPYVGGVEHAHVLLPLLGTLCTVEEICVRDKAVESLCRIGSQMRESDLIDWFVSLVKFAATIEPAFLKTDIMTMFEDLTQDDEDSVRLLAVEGCAALGKLLDPQDRVAHILPVIVNFSQDKSWRVRYMVANQLYELCEAVGPETSRKDLVPSYVRLLRDNEAEVRIAAAGKATKFSQILSPELSLQHILPSVKELSSDSSQHVRSALASVIMGMAPVLGKDATIEHLLPIFLSLLKDEFPDVRLNIISKLDQVNQVIGIDLLSQSLLPAIVELAEDRHWRVRLAIIEYTPMLASQLGVGFFDDKLGTLCMQWLQDEVYSIRDAAANNLKRLAEELGPEWAMQHIIPQVLGVINNSHYLYRMAILRAISLLAPVMGSEITCSKLLPVVITVAKDRVPNVKFNVAKVLQSLIPVVDQSVAEKMIRSSLVELAEDPDVDVRFYASQALQSIDGVMMSS

(19) United States
(12) Patent Application Publication
(10) Pub. No.: US 2021/0238615 A1
(43) Pub. Date: Aug. 5, 2021

(54) PLANT DISEASE RESISTANCE TO PHYTOPHTHORA

(71) Applicant: The Regents of the University of California, Oakland, CA (US)

(72) Inventors: Wenbo Ma, Riverside, CA (US); Tung Kuan, Riverside, CA (US)

(21) Appl. No.: 16/777,023

(22) Filed: Jan. 30, 2020

(51) Int. Cl. C12N 15/82; C12N 9/18

(52) U.S. Cl. CPC C12N 15/8218; C12N 9/18

(57) ABSTRACT

Plants having reduced susceptibility to Phytophthora from modifying or knocking out a native PP2A subunit A.

### CROSS-REFERENCES TO RELATED APPLICATIONS

The present application claims benefit of priority to U.S. provisional patent application No. 62/801,490, filed Feb. 5, 2019, which is incorporated by reference for all purposes.

### STATEMENT AS TO RIGHTS TO INVENTIONS MADE UNDER FEDERALLY SPONSORED RESEARCH AND DEVELOPMENT

This invention was made with Government support under U.S. Department of Agriculture—National Institute of Food and Agriculture, award #2018-67014-28488, and (2) the National Science Foundation, award #IOS-1758889. The government has certain rights in this invention.

### BACKGROUND OF THE INVENTION

Phytophthora belong to a group of fungus-like and zoospore-forming microorganisms, which are important plant pathogens that cause diseases on a broad range of crop and tree species worldwide. However, the control of Phytophthora diseases remains challenging due to the lack of understanding of their pathogenesis. Phytophthora are successful plant pathogens since they encode hundreds of effectors to suppress plant immune responses. Among them, the PSR2 family effectors are evolutionarily conserved among several Phytophthora species. Both PsPSR2 (encoded by Phytophthora sojae) and PiPSR2 (encoded by Phytophthora infestans) function as RNA silencing suppressors and promote Phytophthora infection in plants. See, e.g., Qiao Y, et al. (2013) Nat Genet 45:330-333; Xiong Q, et al. (2014) Mol Plant Microbe Interact 27:1379-1389; and de Vries S, et al. (2017) Mol Plant Pathol 18:110-124.

### BRIEF SUMMARY OF THE INVENTION

In some embodiments, a plant is provided comprising one or more (e.g., one, two, three) modified native type 2A serine/threonine protein phosphatase (PP2A) subunit A or wherein the plant is knocked out for one or more (e.g., one, two, three) PP2A subunit A, wherein the plant is less susceptible to Phytophthora than a control plant comprising a native PP2A subunit A. In some embodiments, the modified native PP2A subunit A is at least 70, 75, 80, 85, 90, or 95% identical to one or more of SEQ ID NO: 1, 2, 3, 4, 5, 6, 7, 8, 9, 10, or 11. In some embodiments, the native PP2A subunit A is at least 70, 75, 80, 85, 90, or 95% identical to one or more of SEQ ID NO: 1, 2, 3, 4, 5, 6, 7, 8, 9, 10, or 11.

In some embodiments, the plant comprises the modified native type 2A serine/threonine protein phosphatase (PP2A) subunit A. In some embodiments, the modification is a point mutation compared to the native PP2A subunit A. In some embodiments, the modification is a deletion or truncation compared to the native PP2A subunit A.

In some embodiments, the plant is knocked out for a PP2A subunit A.

Also provided is a method of making a plant that is less susceptible to Phytophthora than a control plant comprising a native type 2A serine/threonine protein phosphatase (PP2A) subunit A. In some embodiments, the method comprises, introducing a modification in the native PP2A subunit A to form a modified native PP2A subunit A, or knocking out the native PP2A subunit A in a plant, and following the introducing, testing the plant for susceptibility to Phytophthora. In some embodiments, the modified native PP2A subunit A is at least 70, 75, 80, 85, 90, or 95% identical to one or more of SEQ ID NO: 1, 2, 3, 4, 5, 6, 7, 8, 9, 10, or 11.

In some embodiments, the plant comprises the modified native type 2A serine/threonine protein phosphatase (PP2A) subunit A. In some embodiments, the modification is a point mutation compared to the native PP2A subunit A. In some embodiments, the modification is a deletion or truncation compared to the native PP2A subunit A.

In some embodiments, the method comprises knocking out the native PP2A subunit A in the plant.

### Definitions

An “endogenous” gene or protein sequence refers to a non-recombinant sequence of an organism as the sequence occurs in the organism before human-induced mutation of the sequence. A “mutated” sequence refers to a human-altered sequence. Examples of human-induced mutation include exposure of an organism to a high dose of chemical, radiological, or insertional mutagen for the purposes of selecting mutants, as well as recombinant alteration of a sequence. Examples of human-induced recombinant alterations can include, e.g., fusions, insertions, deletions, and/or changes to the sequence.

The term “promoter” refers to regions or sequence located upstream and/or downstream from the start of transcription and which are involved in recognition and binding of RNA polymerase and other proteins to initiate transcription. A “plant promoter” is a promoter capable of initiating transcription in plant cells. A plant promoter can be, but does not have to be, a nucleic acid sequence originally isolated from a plant.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, shoot vegetative organs/structures (e.g. leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A polynucleotide or polypeptide sequence is “heterologous to” an organism or a second sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety).

“Recombinant” refers to a human manipulated polynucleotide or a copy or complement of a human manipulated polynucleotide. For instance, a recombinant expression cassette comprising a promoter operably linked to a second polynucleotide may include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology, Volumes 1-3, John Wiley & Sons, Inc. (1994-1998)). In another example, a recombinant expression cassette may comprise polynucleotides combined in such a way that the polynucleotides are extremely unlikely to be found in nature. For instance, human manipulated restriction sites or plasmid vector sequences may flank or separate the promoter from the second polynucleotide. Polynucleotides can be manipulated in many ways and are not limited to the examples above.

A “transgene” is used as the term is understood in the art and refers to a heterologous nucleic acid introduced into a cell by human molecular manipulation of the cell's genome (e.g., by molecular transformation). Thus a “transgenic plant” is a plant comprising a transgene, i.e., is a genetically-modified plant. The transgenic plant can be the initial plant into which the transgene was introduced as well as progeny thereof whose genome contain the transgene.

An “expression cassette” refers to a nucleic acid construct, which when introduced into a host cell (e.g., a plant cell), results in transcription and/or translation of a RNA or polypeptide, respectively. An expression cassette can result in transcription without translation, for example, when an siRNA or other non-protein encoding RNA is transcribed.

The term “substantial identity” of polynucleotide sequences means that a polynucleotide comprises a sequence that has at least 25% sequence identity to a designated reference sequence. Alternatively, percent identity can be any integer from 70% to 100%, for example, at least: 70%, 75%, 80%, 85%, 90%, 95%, or 99% compared to a reference sequence using the programs described herein; preferably BLAST using standard parameters, as described below. One of skill will recognize that the percent identity values above can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 70%. Percent identity of polypeptides can be any integer from 70% to 100%, for example, at least 70%, 75%, 80%, 85%, 90%, 95%, or 99%. In some embodiments, polypeptides that are “substantially similar” share sequences as noted above except that residue positions that are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, aspartic acid-glutamic acid, and asparagine-glutamine.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions, such as from 20 to 600, usually about 50 to about 200, more usually about 100 to about 150, in which a sequence may be compared to a reference sequence of the same number of contiguous positions after the two sequences are optimally aligned. If no range is provided, the comparison window is the entire length of the reference sequence. Methods of alignment of sequences for comparison are well-known in the art. Optimal alignment of sequences for comparison can be conducted e.g., by the local homology algorithm of Smith and Waterman, Adv. Appl. Math. 2:482, 1981; by the homology alignment algorithm of Needleman and Wunsch, J. Mol. Biol. 48:443, 1970; by the search for similarity method of Pearson and Lipman, Proc. Nat'l. Acad. Sci. USA 85:2444, 1988; by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

An algorithm that is suitable for determining percent sequence identity and sequence similarity is the BLAST algorithm, which is described in Altschul, S. F. et al., J. Mol. Biol. 215:403-410, 1990. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, S. F. et al., supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915, 1989), alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Nat'l. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01, and more preferably less than about 0.001.

### BRIEF DESCRIPTION OF THE DRAWINGS

FIG. 1 depicts a phylogenetic tree of different PP2A subunit A from various species.

FIG. 2 depicts data showing the pdf1 mutant of Arabidopsis showed enhanced resistance against Phytophthora capsici.

FIG. 3 shows data to show RCN1396-588 fragment interacts with PP2A C subunit, but not PSR2.

FIG. 4 shows data indicating PiPSR2 interacts with RCN1 and PDF1.

### DETAILED DESCRIPTION OF THE INVENTION

The inventors have discovered that the Phytophthora effector protein (a virulence factor shown to enhance plant susceptibility to Phytophthora infection) called PSR2 interacts with the plant serine/threonine protein phosphatase 2A (PP2A) subunit A. PP2A functions as a tripartite complex which contains three subunits: A, B and C. The PP2A A subunit is a scaffold that combines a B subunit (a regulatory subunit that recruits various substrates) and a C subunit (a catalytic subunit that has dephosphorylation enzymatic activity) subunit. Subunit A is required for the formation of a functional phosphatase complex. The PP2A complexes are highly conserved in all eukaryotic organisms. In Arabidopsis, there are three A subunits, RCN1, PP2A A2 (aka PDF1) and PP2A A3 (aka PDF2). Phytophthora PSR2 interacts strongly with PDF1, slightly weaker with RCN1, but does not interact with PDF2. The interactions of PSR2 with PDF1 and RCN1 has been confirmed by yeast two hybrid and pull-down assays. pdf1 null mutants have been generated in Arabidopsis and were more resistant to Phytophthora infection.

Accordingly, the present disclosure provides plants have reduced susceptibility to Phytophthora (including but not limited to Phytophthora sojae, Phytophthora infestans, or Phytophthora capsici) resulting from the knockout or mutation of PP2A subunit A in the plants. The plant's susceptibility is “reduced” compared to a control plant (e.g., an otherwise equivalent plant having a native PP2A subunit A corresponding to the subunit A that is knocked out or mutated in the plant having reduced susceptibility). Also provided is methods of making such plants having reduced susceptibility to Phytophthora.

Plants having reduced susceptibility to Phytophthora can be knocked out for a PP2A subunit A or the PP2A subunit A can be mutated such that it no longer interacts with Phytophthora PSR2.

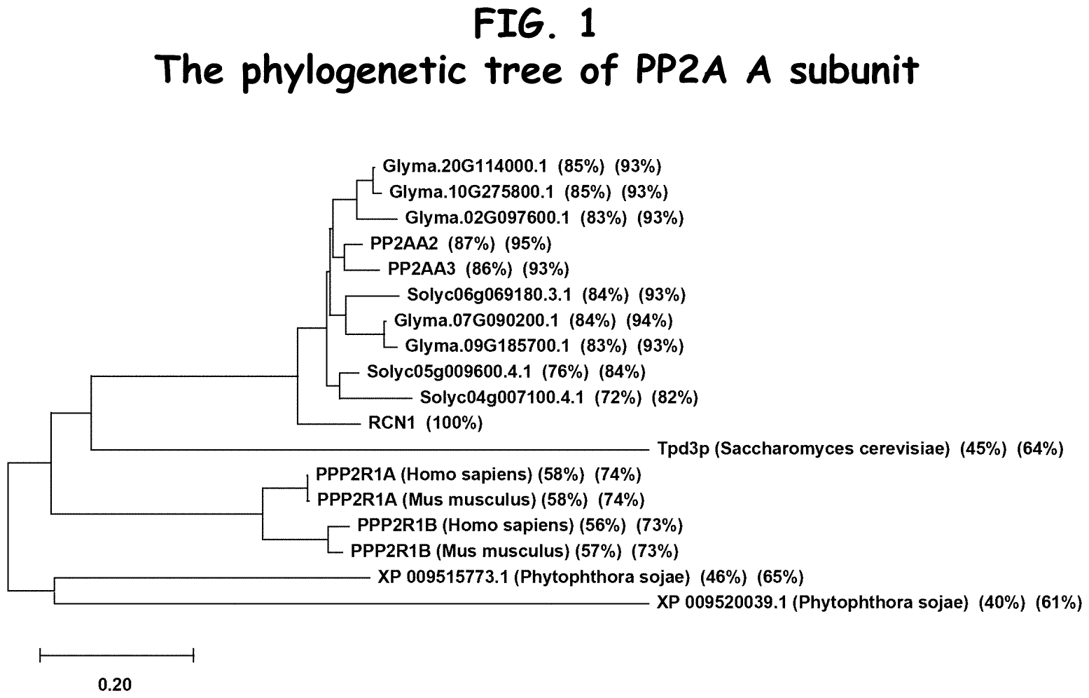

It is believed any plant PP2A subunit A that interacts with Phytophthora PSR2 can be knocked out or mutated to reduce susceptibility to Phytophthora. For example, in some embodiments, the native PP2A subunit A mutated or knocked out in a plant is identical or substantially identical (e.g., at least 70, 75, 80, 85, 90, or 95% identical) to any one of SEQ ID NO:1, 2, 3, 4, 5, 6, 7, 8, 9, 10, or 11. PP2A subunit A can be readily identified in many plant species in view of known genome sequences and the conserved nature of the protein. See, e.g., FIG. 1.

In some embodiments, the PP2A subunit A is knocked out in the plant. “Knocked out” means that the plant does not make the particular PP2A subunit A protein that binds the Phytophthora PSR2 protein. Knockouts can be achieved in a variety of ways. For the purposes of this document, a knock out can be achieved by a deletion of all or a substantial part (e.g., majority) or the coding sequence for the PP2A subunit A such that the protein produced, if any, does not interact with the Phytophthora PSR2. Alternatively a knock out can be achieved by introduction of a mutation that prevents translation or transcription (e.g., a mutation that introduces a stop codon early in the coding sequence or that disrupts transcription). A knock out can also be achieved by silencing or other suppression methods, e.g., such that the plant expresses substantially less of the PP2A subunit A protein (e.g., less than 50, 25, 10, 5, or 1% of native expression).

In some embodiments, the mutation introduced into the native PP2A subunit A protein is a single amino acid change that reduces or eliminates binding of PP2A subunit A to Phytophthora PSR2. Alternatively, the mutation can include any number (e.g., 1, 2, 3, 4, 5, 6, 7, 8, 9, or 10 or more) of amino acid changes, deletions or insertions that reduce or eliminate binding of PP2A subunit A to Phytophthora PSR2.

Methods for introducing genetic mutations into plant genes and selecting plants with desired traits are well known and can be used to introduce mutations or to knock out a PP2A subunit A protein. For instance, seeds or other plant material can be treated with a mutagenic insertional polynucleotide (e.g., transposon, T-DNA, etc.) or chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used. Plants having mutated a PP2A subunit A protein can then be identified, for example, by phenotype or by molecular techniques.

Modified protein chains can also be readily designed utilizing various recombinant DNA techniques well known to those skilled in the art and described for instance, in Sambrook et al., supra. Hydroxylamine can also be used to introduce single base mutations into the coding region of the gene (Sikorski et al., Meth. Enzymol., 194:302-318 (1991)). For example, the chains can vary from the naturally occurring sequence at the primary structure level by amino acid substitutions, additions, deletions, and the like. These modifications can be used in a number of combinations to produce the final modified protein chain.

Alternatively, homologous recombination can be used to induce targeted gene modifications or knockouts by specifically targeting the PP2A subunit A gene in vivo (see, generally, Grewal and Klar, Genetics, 146:1221-1238 (1997) and Xu et al., Genes Dev., 10:2411-2422 (1996)). Homologous recombination has been demonstrated in plants (Puchta et al., Experientia, 50:277-284 (1994); Swoboda et al., EMBO 1, 13:484-489 (1994); Offringa et al., Proc. Natl. Acad. Sci. USA, 90:7346-7350 (1993); and Kempin et al., Nature, 389:802-803 (1997)).

In applying homologous recombination technology to a PP2A subunit A protein gene, mutations in selected portions of PP2A subunit A gene sequences (including 5′ upstream, 3′ downstream, and intragenic regions) can be made in vitro and then introduced into the desired plant using standard techniques. Since the efficiency of homologous recombination is known to be dependent on the vectors used, use of dicistronic gene targeting vectors as described by Mountford et al., Proc. Natl. Acad. Sci. USA, 91:4303-4307 (1994); and Vaulont et al., Transgenic Res., 4:247-255 (1995) are conveniently used to increase the efficiency of selecting for altered PP2A subunit A protein gene expression in transgenic plants. The mutated gene will interact with the target wild-type gene in such a way that homologous recombination and targeted replacement of the wild-type gene will occur in transgenic plant cells, resulting in suppression of PP2A subunit A protein activity.

Any of a number of genome editing proteins known to those of skill in the art can be used to mutate or knock out the PP2A subunit A protein. The particular genome editing protein used is not critical, so long as it provides site-specific mutation of a desired nucleic acid sequence. Exemplary genome editing proteins include targeted nucleases such as engineered zinc finger nucleases (ZFNs), transcription-activator like effector nucleases (TALENs), and engineered meganucleases. In addition, systems which rely on an engineered guide RNA (a gRNA) to guide an endonuclease to a target cleavage site can be used. The most commonly used of these systems is the CRISPR/Cas system with an engineered guide RNA to guide the Cas-9 endonuclease to the target cleavage site.

CRISPR (Clustered Regularly Interspaced Short Palindromic Repeats)/Cas (CRISPR-associated) system, are adaptive defense systems in prokaryotic organisms that cleave foreign DNA. CRISPR loci in microbial hosts contain a combination of CRISPR-associated (Cas) genes as well as non-coding RNA elements which determine the specificity of the CRISPR-mediated nucleic acid cleavage. Three types (I-III) of CRISPR systems have been identified across a wide range of bacterial hosts. In the typical system, a Cas endonuclease (e.g., Cas9) is guided to a desired site in the genome using small RNAs that target sequence-specific single- or double-stranded DNA sequences. The CRISPR/Cas system has been used to induce site-specific mutations in plants (see Miao et al. 2013 Cell Research 23:1233-1236).

The basic CRISPR system uses two non-coding guide RNAs (crRNA and tracrRNA) which form a crRNA:tracrRNA complex that directs the nuclease to the target DNA via Wastson-Crick base-pairing between the crRNA and the target DNA. Thus, the guide RNAs can be modified to recognize any desired target DNA sequence. More recently, it has been shown that a Cas nuclease can be targeted to the target gene location with a chimeric single-guide RNA (sgRNA) that contains both the crRNA and tracRNA elements. It has been shown that Cas9 can be targeted to desired gene locations in a variety of organisms with a chimeric sgRNA (Cong et al. 2013 Science 339:819-23).

Zinc finger nucleases (ZFNs) are engineered proteins comprising a zinc finger DNA-binding domain fused to a nucleic acid cleavage domain, e.g., a nuclease. The zinc finger binding domains provide specificity and can be engineered to specifically recognize any desired target DNA sequence. For a review of the construction and use of ZFNs in plants and other organisms, see Urnov et al. 2010 Nat Rev Genet. 11(9):636-46.

Transcription activator like effectors (TALEs) are proteins secreted by certain species of Xanthomonas to modulate gene expression in host plants and to facilitate bacterial colonization and survival. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site have been found. The simple relationship between amino acid sequence and DNA recognition of the TALE binding domain allows for the design DNA binding domains of any desired specificity.

TALEs can be linked to a non-specific DNA cleavage domain to prepare genome editing proteins, referred to as TALENs. As in the case of ZFNs, a restriction endonuclease, such as FokI, can be conveniently used. For a description of the use of TALENs in plants, see Mahfouz et al. 2011 Proc Natl Acad Sci USA. 108:2623-8 and Mahfouz 2011 G M Crops. 2:99-103.

Meganucleases are endonucleases that have a recognition site of 12 to 40 base pairs. As a result, the recognition site occurs rarely in any given genome. By modifying the recognition sequence through protein engineering, the targeted sequence can be changed and the nuclease can be used to cleave a desired target sequence. (See Seligman, et al. 2002 Nucleic Acids Research 30: 3870-9 WO06097853, WO06097784, WO04067736, or US20070117128).

In addition to the methods described above, other methods for introducing genetic mutations into plant genes and selecting plants with desired traits are known. For instance, seeds or other plant material can be treated with a mutagenic chemical substance, according to standard techniques. Such chemical substances include, diethyl sulfate, ethylene imine, ethyl methanesulfonate (EMS) and N-nitroso-N-ethylurea. Alternatively, ionizing radiation from sources such as, X-rays or gamma rays can be used.

Also provided are methods of suppressing PP2A subunit A expression or activity in a plant using expression cassettes that transcribe PP2A subunit A RNA molecules (or fragments thereof) that inhibit endogenous PP2A subunit A expression or activity in a plant cell. Suppressing or silencing gene function refers generally to the suppression of levels PP2A subunit A mRNA or PP2A subunit A protein expressed by the endogenous PP2A subunit A gene and/or the level of the PP2A subunit A protein functionality in a cell. The terms do not require specific mechanism and could include RNAi (e.g., short interfering RNA (siRNA) and microRNA (miRNA)), anti-sense, cosuppression, viral-suppression, hairpin suppression, stem-loop suppression, and the like.

A number of methods can be used to suppress or silence gene expression in a plant. The ability to suppress gene function in a variety of organisms, including plants, using double stranded RNA is well known. Expression cassettes encoding RNAi typically comprise a polynucleotide sequence at least substantially identical to the target gene linked to a complementary polynucleotide sequence. The sequence and its complement are often connected through a linker sequence that allows the transcribed RNA molecule to fold over such that the two sequences hybridize to each other.

RNAi (e.g., siRNA, miRNA) appears to function by base-pairing to complementary RNA or DNA target sequences. When bound to RNA, the inhibitory RNA molecules trigger either RNA cleavage or translational inhibition of the target sequence. When bound to DNA target sequences, it is thought that inhibitory RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides in length that are processed from longer precursor transcripts that form stable hairpin structures.

In addition, antisense technology can be conveniently used. To accomplish this, a nucleic acid segment at least substantially identical to the desired gene is cloned and operably linked to a promoter such that the antisense strand of RNA will be transcribed. The expression cassette is then transformed into a plant and the antisense strand of RNA is produced. In plant cells, it has been suggested that antisense RNA inhibits gene expression by preventing the accumulation of mRNA which encodes the protein of interest.

Another method of suppression is sense suppression. Introduction of expression cassettes in which a nucleic acid is configured in the sense orientation with respect to the promoter has been shown to be an effective means by which to block the transcription of target genes.

For these techniques, the introduced sequence in the expression cassette need not have absolute identity to the target gene. In addition, the sequence need not be full length, relative to either the primary transcription product or fully processed mRNA. One of skill in the art will also recognize that using these technologies families of genes can be suppressed with a transcript. For instance, if a transcript is designed to have a sequence that is conserved among a family of genes, then multiple members of a gene family can be suppressed. Conversely, if the goal is to only suppress one member of a homologous gene family, then the transcript should be targeted to sequences with the most variance between family members.

Gene expression can also be inactivated using recombinant DNA techniques by transforming plant cells with constructs comprising transposons or T-DNA sequences. Mutants prepared by these methods are identified according to standard techniques. For instance, mutants can be detected by PCR or by detecting the presence or absence of PP2A subunit A mRNA, e.g., by northern blots or reverse transcription PCR (RT-PCR).

Catalytic RNA molecules or ribozymes can also be used to inhibit expression of embryo-specific genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA cleaving activity upon them, thereby increasing the activity of the constructs. The design and use of target RNA-specific ribozymes is well known.

The recombinant construct encoding a genome editing protein or a nucleic acid that suppresses PP2A subunit A expression may be introduced into the plant cell using standard genetic engineering techniques, well known to those of skill in the art. In the typical embodiment, recombinant expression cassettes can be prepared according to well-known techniques. In the case of CRISPR/Cas nuclease, the expression cassette may transcribe the guide RNA, as well.

Such plant expression cassettes typically contain the polynucleotide operably linked to a promoter (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A number of promoters can be used. A plant promoter fragment can be employed which will direct expression of the desired polynucleotide in all tissues of a plant. Such promoters are referred to herein as “constitutive” promoters and are active under most environmental conditions and state of development or cell differentiation. Examples of constitutive promoters include the cauliflower mosaic virus (CaMV) 35S transcription initiation region.

Alternatively, the plant promoter can direct expression of the polynucleotide under environmental control. Such promoters are referred to here as “inducible” promoters. Environmental conditions that may affect transcription by inducible promoters include biotic stress, abiotic stress, saline stress, drought stress, pathogen attack, anaerobic conditions, cold stress, heat stress, hypoxia stress, or the presence of light.

In addition, chemically inducible promoters can be used. Examples include those that are induced by benzyl sulfonamide, tetracycline, abscisic acid, dexamethasone, ethanol or cyclohexenol.

Examples of promoters under developmental control include promoters that initiate transcription only, or preferentially, in certain tissues such as leaves, roots, fruit, seeds, or flowers. These promoters are sometimes called tissue-preferred promoters. The operation of a promoter may also vary depending on its location in the genome. Thus, a developmentally regulated promoter may become fully or partially constitutive in certain locations. A developmentally regulated promoter can also be modified, if necessary, for weak expression.

Methods for transformation of plant cells are well known in the art, and the selection of the most appropriate transformation technique for a particular embodiment of the invention may be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens mediated transformation. Transformation means introducing a nucleotide sequence in a plant in a manner to cause stable or transient expression of the sequence.

In some embodiments, in planta transformation techniques (e.g., vacuum-infiltration, floral spraying or floral dip procedures) are used to introduce the expression cassettes of the invention (typically in an Agrobacterium vector) into meristematic or germline cells of a whole plant. Such methods provide a simple and reliable method of obtaining transformants at high efficiency while avoiding the use of tissue culture. (see, e.g., Bechtold et al. 1993 C. R. Acad. Sci. 316:1194-1199; Chung et al. 2000 Transgenic Res. 9:471-476; Clough et al. 1998 Plant J. 16:735-743; and Desfeux et al. 2000 Plant Physiol 123:895-904). In these embodiments, seed produced by the plant comprise the expression cassettes encoding the genome editing proteins of the invention. The seed can be selected based on the ability to germinate under conditions that inhibit germination of the untransformed seed.

If transformation techniques require use of tissue culture, transformed cells may be regenerated into plants in accordance with techniques well known to those of skill in the art. The regenerated plants may then be grown, and crossed with the same or different plant varieties using traditional breeding techniques to produce seed, which are then selected under the appropriate conditions.

The expression cassette can be integrated into the genome of the plant cells, in which case subsequent generations will express the encoded proteins. Alternatively, the expression cassette is not integrated into the genome of the plants cell, in which case the encoded protein is transiently expressed in the transformed cells and is not expressed in subsequent generations.

In some embodiments, the genome editing protein itself, is introduced into the plant cell. In these embodiments, the introduced genome editing protein is provided in sufficient quantity to modify the cell but does not persist after a contemplated period of time has passed or after one or more cell divisions. In such embodiments, no further steps are needed to remove or segregate away the genome editing protein and the modified cell.

In these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

Any plant that expresses a native PP2A subunit A protein can be modified as described herein to have reduced susceptibility to Phytophthora. Exemplary plants include species from the genera Arachis, Asparagus, Atropa, Aven, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malta, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and Zea.

Determination of relative plant susceptibility to Phytophthora can be performed as known in the art. For example, test plants and control plants (e.g., plant having a modified PP2A subunit A described herein and a control native plat) can be contacted with the same number of Phytophthora zoospores or hyphae and then monitored for the development of disease symptoms.

The ability of a modified PP2A subunit A protein to interact (e.g., bind) to a Phytophthora PSR2 protein can be determined by yeast two-hybrid assays or using a pulldown assay. Pull-down assays are a form of affinity purification and are similar to immunoprecipitation, except that a “bait” protein is used instead of an antibody. See, e.g., Einarson M B, Orlinick J R (2002) Identification of Protein-Protein Interactions with Glutathione S-Transferase Fusion Proteins. In: Protein-Protein Interactions: A Molecular Cloning Manual. Cold Spring Harbor (N.Y.): Cold Spring Harbor Laboratory Press. pp 37-57; Einarson M B (2001) Detection of Protein-Protein Interactions Using the GST Fusion Protein Pulldown Technique. In: Molecular Cloning: A Laboratory Manual, 3rd Edition. Cold Spring Harbor (N.Y.): Cold Spring Harbor Laboratory Press. pp 18.55-18.59; and Vikis H G, Guan K-L (2004) Glutathione-S-Transferase-Fusion Based Assays for Studying Protein-Protein Interactions. In: Fu H (editor), Protein-Protein Interactions, Methods and Applications, Methods in Molecular Biology, 261. Totowa (N.J.): Humana Press. pp 175-186. The particular PSR2 protein used in a binding assay will generally be the native Phytophthora PSR2 protein, optionally comprising a fusion partner (e.g., GST) for manipulation of the protein in the binding assay. Exemplary PSR2 proteins include but are not limited to PsPSR2 (encoded by Phytophthora sojae) and PiPSR2 (encoded by Phytophthora infestans).

### Example

The following examples are offered to illustrate, but not to limit the claimed invention.

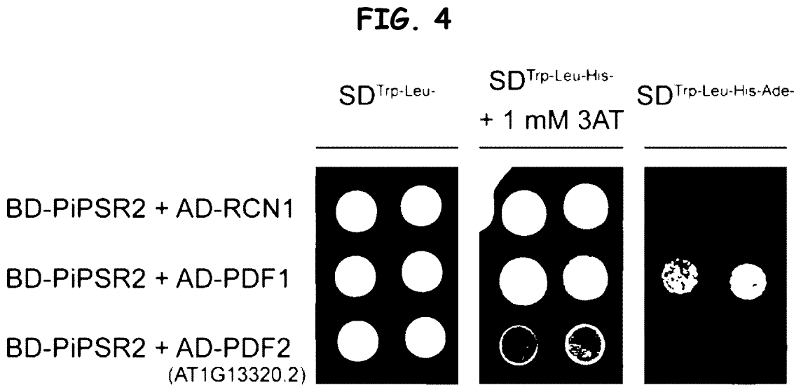

We found that the Phytophthora effector protein (a virulence factor that we have previously shown to enhance plant susceptibility to Phytophthora infection) called PSR2 interacts with the Arabidopsis serine/threonine protein phosphatase 2A (PP2A) subunit A. The PP2A complexes are highly conserved in all eukaryotic organisms. In Arabidopsis, there are three A subunits, RCN1, PP2A A2 (aka PDF1) and PP2A A3 (aka PDF2). PSR2 interacts strongly with PDF1, slightly weaker with RCN1, but does not interact with PDF2. The interactions of PSR2 with PDF1 and RCN1 has been confirmed by yeast two hybrid (FIG. 4) and pull-down assays.

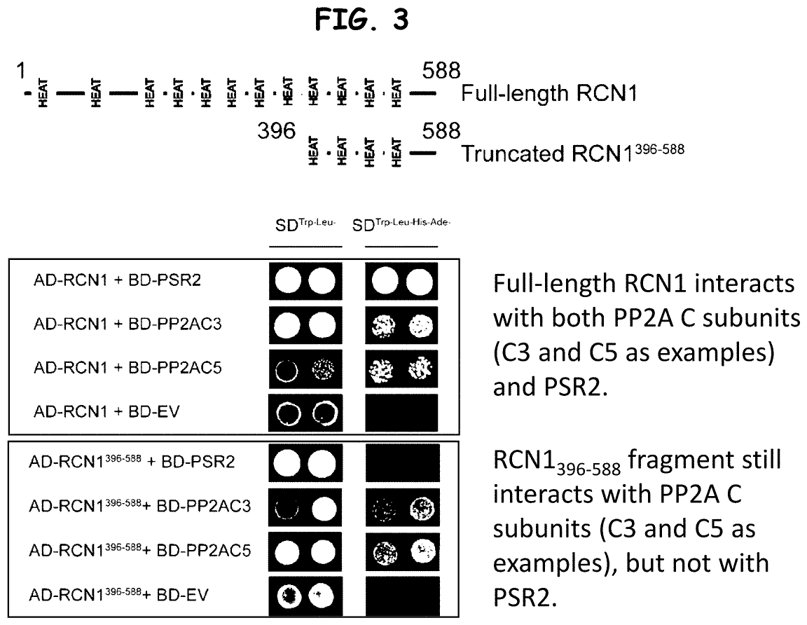

Analysis using RCN1 truncations indicates that PSR2 interacts with the portion of RCN1 that would interact with an endogenous PP2A B subunit. A truncated RCN1 (containing the C-terminal 396-588 aa) that no longer interacts with PSR2 can still interacts with the C subunit, indicating that PSR2 and the C subunit do not interact with the same location within the A subunit. See, FIG. 3.

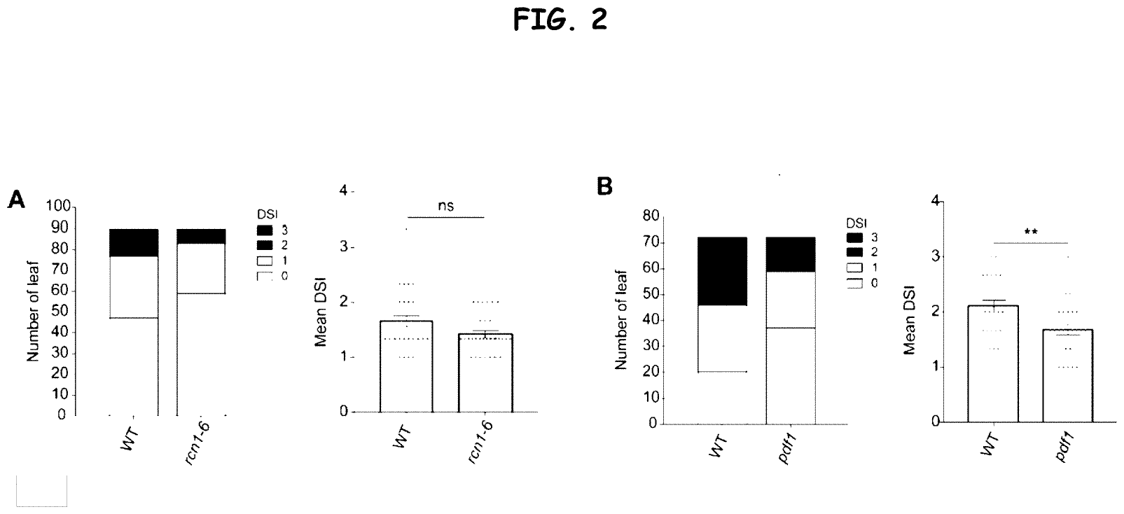

Both rcn1 and pdf1 null mutants were analyzed in Arabidopsis and the pdf1 mutant exhibited significant resistance to Phytophthora infection. FIG. 2 depicts data from the pdf1 null mutant. These results indicate that PDF1 are “helpers” to Phytophthora infection, and hence may be considered as susceptibility genes in plants. The rcn1 mutant showed moderate resistance that was not statistically significant. An Arabidopsis mutant with both rcn1 and pdf1 knocked out is developmentally defective, so we did not test this mutant on disease susceptibility. Single mutants of rcn1 or pdf1 do not show obvious developmental deficiency.

The following protocol was used to determine plant Phytophthora susceptibility:
1. Four-week-old Arabidopsis plants were used for inoculation by the Phytophthora capsici strain LT263.
2. Each plant contributes 3 detached leaves (usually the 4th, 5th, and 6th leaf from the top) for examining susceptibility. 12-30 adult leaves from 4-10 plants of each genotype were placed up-side-down on the 0.8% water agar plate, and each leaf was inoculated with 10 μL of zoospore suspension (approximate 10⁵ zoospores/mL) as a droplet on the abaxial side.
3. The plates were wrapped with Parafilm to maintain high humidity and incubated in the dark at room temperature for 2-4 days. Disease severity was evaluated at 2, 3 and 4 days post inoculation.
4. Using disease severity index (DSI) with the scale from 0 to 3 to evaluate susceptibility level of each leaf. Leaves with no visible disease symptoms or only small necrotic flecks restricted to the inoculation area were scored as DSI=0. Leaves with water soaking-like lesion spreading from the inoculation spot but only covering less than 50% of the leaf were scored as DSI=1. Leaves with water soaking-like lesion covering 50% to 75% of the leaf were scored as DSI=2. Leaves that were completely wilted or had water soaking-like lesion fully covering the leaf were scored as DSI=3. Mean DSI in each genotype was analyzed using the equation below and data from three independent experiments are presented as stacked bar graphs.

$$Mean DSI of each plant = \frac{\{ {\sum }_{index no .} [ ( index no . + 1 ) \times ( amount of leaves in each index ) ] \}}{Total amount of leaves ( 3 leaves each plant )}$$

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

SEQUENCES
>RCN1
SEQ ID NO: 1
MAMVDEPLYP IAVLIDELKN DDIQLRLNSI RRLSTIARAL GEERTRKELI
PFLSENSDDD DEVLLAMAEE LGVFIPFVGG IEFAHVLLPP LESLCTVEET
CVREKAVESL CKIGSQMKEN DLVESFVPLV KRLAGGEWFA ARVSACGIFH
VAYQGCTDVL KTELRATYSQ LCKDDMPMVR RAAASNLGKF ATTVESTFLI
AEIMTMFDDL TKDDQDSVRL LAVEGCAALG KLLEPQDCVA RILPVIVNFS
QDKSWRVRYM VANQLYELCE AVGPDCTRTD LVPAYVRLLR DNEAEVRIAA
AGKVTKFCRL LNPELAIQHI LPCVKELSSD SSQHVRSALA SVIMGMAPIL
GKDSTIEHLL PIFLSLLKDE FPDVRLNIIS KLDQVNQVIG IDLLSQSLLP
AIVELAEDRH WRVRLAIIEY VPLLASQLGI GFFDDKLGAL CMQWLQDKVY
SIREAAANNL KRLAEEFGPE WAMQHLVPQV LDMVNNPHYL HRMMVLRAIS
LMAPVMGSEI TCSKFLPVVV EASKDRVPNI KFNVAKLLQS LIPIVDQSVV
DKTIRQCLVD LSEDPDVDVR YFANQALNSI DGSTAAQS
>PP2A A2 (PDF1)
SEQ ID NO: 2
MSMIDEPLYP IAVLIDELKN DDIQLRLNSI RRLSTIARAL GEERTRKELI
PFLSENNDDD DEVLLAMAEE LGVFIPYVGG VEYAHVLLPP LETLSTVEET
CVREKAVESL CRVGSQMRES DLVDHFISLV KRLAAGEWFT ARVSACGVFH
IAYPSAPDML KTELRSLYTQ LCQDDMPMVR RAAATNLGKF AATVESAHLK
TDVMSMFEDL TQDDQDSVRL LAVEGCAALG KLLEPQDCVQ HILPVIVNFS
QDKSWRVRYM VANQLYELCE AVGPEPTRTE LVPAYVRLLR DNEAEVRIAA
AGKVTKFCRI LNPEIAIQHI LPCVKELSSD SSQHVRSALA SVIMGMAPVL
GKDATIEHLL PIFLSLLKDE FPDVRLNIIS KLDQVNQVIG IDLLSQSLLP
AIVELAEDRH WRVRLAIIEY IPLLASQLGV GFFDDKLGAL CMQWLQDKVH
SIRDAAANNL KRLAEEFGPE WAMQHIVPQV LEMVNNPHYL YRMTILRAVS
LLAPVMGSEI TCSKLLPVVM TASKDRVPNI KFNVAKVLQS LIPIVDQSVV
EKTIRPGLVE LSEDPDVDVR FFANQALQSI DNVMMSS
>PP2A A3 (PDF2)
SEQ ID NO: 3
MSMVDEPLYP IAVLIDELKN DDIQRRLNSI KRLSIIARAL GEERTRKELI
PFLSENNDDD DEVLLAMAEE LGGFILYVGG VEYAYVLLPP LETLSTVEET
CVREKAVDSL CRIGAQMRES DLVEHFTPLA KRLSAGEWFT ARVSACGIFH
IAYPSAPDVL KTELRSIYGQ LCQDDMPMVR RAAATNLGKF AATIESAHLK
TDIMSMFEDL TQDDQDSVRL LAVEGCAALG KLLEPQDCVA HILPVIVNFS
QDKSWRVRYM VANQLYELCE AVGPEPTRTD LVPAYARLLC DNEAEVRIAA
AGKVTKFCRI LNPELAIQHI LPCVKELSSD SSQHVRSALA SVIMGMAPVL
GKDATIEHLL PIFLSLLKDE FPDVRLNIIS KLDQVNQVIG IDLLSQSLLP
AIVELAEDRH WRVRLAIIEY IPLLASQLGV GFFDEKLGAL CMQWLQDKVH
SIREAAANNL KRLAEEFGPE WAMQHIVPQV LEMINNPHYL YRMTILRAVS
LLAPVMGSEI TCSKLLPAVI TASKDRVPNI KFNVAKMMQS LIPIVDQAVV
ENMIRPCLVE LSEDPDVDVR YFANQALQSI DNVMMSS
>Glyma.20G114000.1
SEQ ID NO: 4
MADEPLYPIAVLIDELKNDDIQLRLNSIRRLSTIARALGEERTRRELIPFLSENNDDDDEVL
LAMAEELGVFIPYVGGVEHASVLLPPLETLCTVEETCVRDKAVESLCRIGSQMRESDLVE
YYIPLVKRLAAGEWFTARVSACGLFHIAYPSAPETSKTELRSIYSQLCQDDMPMVRRSA
ASNLGKFAATVEYAHLKADVMSIFDDLTQDDQDSVRLLAVEGCAALGKLLEPQDCVA
HILPVIVNFSQDKSWRVRYMVANQLYELCEAVGPEPTRTELVPAYVRLLRDNEAEVRIA
AAGKVTKFCRILNPDLAIQHILPCVKELSSDSSQHVRSALASVIMGMAPVLGKEATIEQL
LPIFLSLLKDEFPDVRLNIISKLDQVNQVIGIDLLSQSLLPAIVELAEDRHWRVRLAIIEYIP
LLASQLGVRFFDDKLGALCMQWLQDKVHSIREAAANNLKRLAEEFGPEWAMQHIIPQV
LEMNNNPHYLYRMTILRAISLLAPVMGPEITCSNLLPVVLAASKDRVPNIKFNVAKVLES
IFPIVDQSVVEKTIRPCLVELSEDPDVDVRFFSNQALQAIDHVMMSC
>Glyma.10G275800.1
SEQ ID NO: 5
MADEPLYPIAVLIDELKNDDIQLRLNSIRRLSTIARALGEERTRRELIPFLSENNDDDDEVL
LAMAEELGVFIPYVGGVEHASVLLPPLETLCTVEETCVRDKAAESLCRIGSQMRESDLVE
YYIPLVKRLAAGEWFTARVSACGLFHIAYPSAPETSKTELRSIYSQLCQDDMPMVRRSA
ASNLGKFAATVEYAHLKADLMSIFDDLTQDDQDSVRLLAVEGCAALGKLLEPQDCVAH
ILPVIVNFSQDKSWRVRYMVANQLYELCEAVGPEPTRTELVPAYVRLLRDNEAEVRIAA
AGKVTKFCRILNPDLSIQHILSCVKELSSDSSQHVRSALASVIMGMAPVLGKEATIEQLLP
IFLSLLKDEFPDVRLNIISKLDQVNQVIGIDLLSQSLLPAIVELAEDRHWRVRLAIIEYIPLL
ASQLGVSFFDDKLGALCMQWLQDKVHSIREAAANNLKRLAEEFGPEWAMQHIIPQVLE
MNNNPHYLYRMTILRAISLLAPVMGPEITCSNLLPVVVAASKDRVPNIKFNVAKVLESIF
PIVDQSVVEKTIRPCLVELSEDPDVDVRFFSNQALQAIDHVMMSS
>Glyma.07G090200.1
SEQ ID NO: 6
MAMVDQPLYPIAVLIDELKNEDIQLRLNSIRRLSTIARALGEDRTRKELIPFLSENNDDDD
EVLLAMAEELGVFIPYVGGVDHANVLLPPLETLCTVEETCVRDKSVESLCRIGAQMREQ
DLVEHFIPLVKRLAAGEWFTARVSSCGLFHIAYPSAPESVKTELRAIYGQLCQDDMPMV
RRSAATNLGKFAATVEAPHLKSDEVISVFEDLTQDDQDSVRLLAVEGCAALGKLLEPQD
CVAHILPVIVNFSQDKSWRVRYMVANQLYELCEAVGPDPTRSELVPAYVRLLRDNEAE
VRIAAAGKVTKFSRILNPDLAIQHILPCVKELSTDSSQHVRSALASVINTGMAPVLGKDAT
IEQLLPIFLSLLKDEFPDVRLNIISKLDQVNQVIGIDLLSQSLLPAIVELAEDRHWRVRLAII
EYIPLLASQLGVGFFDDKLGALCMQWLKDKVYSIRDAAANNIKRLAEEFGPDWAMQHII
PQVLDMVTDPHYLYRMTILQAISLLAPVLGSEITSSKLLPLVINASKDRVPNIKFNVAKVL
QSLIPIVDQSVVESTIRPCLVELSEDPDVDVRFFASQALQSSDQVKMSS*
>Glyma.02G097600.1
SEQ ID NO: 7
MSMVDEPLYPIAVLIDELKNDDIQLRLNSIRKLSTIARALGEERTRRELIPFLGENNDDDD
EVLLAMAEELGVFIPFVGGVEHAHVLLPPLEMLCTVEETCVRDKAVESLCRIGLQMRES
DLVEYFIPLVKRLASGEWFTARVSSCGLFHIAYPSAPEMSKIELRSMYSLLCQDDMPMV
RRSAASNLGKYAATVEYAHLKADTMSIFEDLTKDDQDSVRLLAVEGCAALGKLLEPQD
CITHILPVIVNFSQDKSWRVRYMVANQLYELCEAVGPEPTRTELVPAYVRLLRDNEAEV
RIAAAGKVTKFCRILNPDLSIQHILPCVKELSTDSLQHVRSALASVINTGMAPVLGKDATIE
QLLPIFLSLLKDEFPDVRLNIISKLDQVNQVIGINLLSQSLLPAIVELAEDRHWRVRLAIIEY
IPLLASQLGVGFFYDKLGALCMQWLQDKVHSIREAAANNLKRLAEEFGPEWAMQHIIPQ
VLEMISNPHYLYRMTILHAISLLAPVMGSEITRSELLPIVITASKDRVPNIKFNVAKVLESI
FPIVDQSVVEKTIRPSLVELSEDPDVDVRFFSNQALHAMDHVMMSS
>Glyma.09G185700.1
SEQ ID NO: 8
MAMVDQPLYPIAVLIDELKNEDIQLRLNSIRRLSTIARALGEDRTRKELIPFLSENNDDDD
EVLLAMAEELGVFIPYVGGVEHANVLLPPLETLCTVEETSVRDKSVESLCRIGAQMREQ
DLVEYLIPLVKRLAAGEWFTARVSSCGLFHIAYPSAPEAVKTELRAIYGQLCQDDMPMV
RRSAATNLGKFAATVEAPHLKSDIMSVFEDLTHDDQDSVRLLAVEGCAALGKLLEPQD
CVAHILPVIVNFSQDKSWRVRYMVANQLYELCEAVGPDPTRSELVPAYVRLLRDNEAE
VRIAAAGKVTKFSRILNPDLAIQHILPCVKELSTDSSQHVRSALASVIMGMAPVLGKDAT
IEQLLPIFLSLLKDEFPDVRLNIISKLDQVNQVIGIDLLSQSLLPAIVELAEDRHWRVRLAII
EYIPLLASQLGVSFFDDKLGALCMQWLKDKVYSIRDAAANNIKRLAEEFGPDWAMQHII
PQVLDMVTDPHYLYRMTILQSISLLAPVLGSETSSSKLLPLVINASKDRVPNIKFNVAKVL
QSLIPIVDQSVVESTIRPCLVELSEDPDVDVRFFASQALQSCDQVKMSS
>Solyc05g009600.4.1
SEQ ID NO: 9
MAEELGVFIPYVGGVEHAHVLLPPLETLCTVEETCVRDKAVESLCRIGSQMRESDLVDW
FVPLVKRLAAGEWFTARVSACGLFHIAYSSAPEMLKAELRSIYSQLCQDDMPMVRRSA
ATNLGKFAATVESAYLKSDIMSIFDDLTQDDQDSVRLLAVEGCAALGKLLEPQDCVAHI
LPVIVNFSQDKSWRVRYMVANQLYELCEAVGPEPTRTDLVPAYVRLLRDNEAEVRIAA
AGKVTKFCRILSPELAIQHILPCVKELSSDSSQHVRSALASVIMGMAPVLGKDATIEHLLP
IFLSLLKDEFPDVRLNIISKLDQVNQVIGIDLLSQSLLPAIVELAEDRHWRVRLAIIEYIPLL
ASQLGIGFFDDKLGALCMQWLQDKVYSIRDAAANNLKRLAEEFGPEWAMQHIIPQVLD
MTTSPHYLYRMTILRSISLLAPVMGSEITCSKLLPVVVTATKDRVPNIKFNVAKVLQSLV
PIVDNSVVEKTIRPSLVELAEDPDVDVRFYANQALQSIDNVMMSG
>Solyc06g069180.3.1
SEQ ID NO: 10
MSAIDEPLYPIAVLIDELKNEDIQLRLNSIRRLSTIARALGEERTRKELIPFLSENNDDDDE
VLLAMAEELGMFIPYVGGVEHARVLLPPLEGLCSVEETCVREKAVESLCKIGSQMKESD
LVESFIPLVKRLATGEWFTARVSSCGLFHIAYPSAPEPLKNELRTIYSQLCQDDMPMVRR
AAATNLGKFAATIEQPHLKTDIMSMFETLTQDDQDSVRLLAVEDCAALGKLLEPKDCV
AQILSVIVNFAQDKSWRVRYMVANQLYDLCEAVGPEATRTDLVPAYVRLLRDNEAEVR
IAAAGKVTKFCRILSPELAIQHILPCVKELSSDSSQHVRSALASVIMGMAPILGKDATIEQ
LLPIFLSLLKDEFPDVRLNIISKLDQVNQVIGIDLLSQSLLPAIVELAEDRHWRVRLAIIEYI
PLLASQLGVGFFDDKLGALCMQWLKDKVYSIRDAAANNVKRLAEEFGPKWAMEHIIPQ
VLDMINDPHYLYRMTILHAISLLAPVLGSEIACSKLLPVIITASKDRVPNIKFNVAKVLQS
VIPIVEQSVVESTIRPCLVELSEDPDVDVRFFANQALQATK
>Solyc04g007100.4.1
SEQ ID NO: 11
NSCTLSKPFDHFCLLSPNTFHFIEINEGNKSSLLNSPDIKGFTSPAAGDTHFRCKGNTIYLS
MAHLLLYPMILDELKNDDIQLRLNSVRRLSSIACQLGEDRTRRELIPFLCRNTDDEDEVL
LAMSEELGGFIPYVGGVEHAHVLLPLLGTLCTVEEICVRDKAVESLCRIGSQMRESDLID
WFVSLVKFAATIEPAELKTDIMTMFEDLTQDDEDSVRLLAVEGCAALGKLLDPQDRVA
HILPVIVNESQDKSWRVRYMVANQLYELCEAVGPETSRKDLVPSYVRLLRDNEAEVRIA
AAGKATKESQILSPELSLQHILPSVKELSSDSSQHVRSALASVIMGMAPVLGKDATIEHLL
PIFLSLLKDEFPDVRLNIISKLDQVNQVIGIDLLSQSLLPAIVELAEDRHWRVRLAIIEYTP
MLASQLGVGFFDDKLGTLCMQWLQDEVYSIRDAAANNLKRLAEELGPEWAMQHIIPQ
VLGVINNSHYLYRMAILRAISLLAPVMGSEITCSKLLPVVITVAKDRVPNVKFNVAKVLQ
SLIPVVDQSVAEKMIRSSLVELAEDPDVDVRFYASQALQSIDGVMMSS

The above examples are provided to illustrate the invention but not to limit its scope. Other variants of the invention will be readily apparent to one of ordinary skill in the art and are encompassed by the appended claims. All publications, databases, internet sources, patents, patent applications, and accession numbers cited herein are hereby incorporated by reference in their entireties for all purposes.

What is claimed is:

1. A plant comprising a modified native type 2A serine/threonine protein phosphatase (PP2A) subunit A or wherein the plant is knocked out for a PP2A subunit A, wherein the plant is less susceptible to Phytophthora than a control plant comprising a native PP2A subunit A.

2. The plant of claim 1, wherein the modified native PP2A subunit A is at least 70, 75, 80, 85, 90, or 95% identical to one or more of SEQ ID NO: 1, 2, 3, 4, 5, 6, 7, 8, 9, 10, or 11.

3. The plant of claim 1, wherein the native PP2A subunit A is at least 70, 75, 80, 85, 90, or 95% identical to one or more of SEQ ID NO: 1, 2, 3, 4, 5, 6, 7, 8, 9, 10, or 11.

4. The plant of claim 1, wherein the plant comprises the modified native type 2A serine/threonine protein phosphatase (PP2A) subunit A.

5. The plant of claim 4, wherein the modification is a point mutation compared to the native PP2A subunit A.

6. The plant of claim 4, wherein the modification is a deletion or truncation compared to the native PP2A subunit A.

7. The plant of claim 1, wherein the plant is knocked out for a PP2A subunit A.

8. A method of making a plant that is less susceptible to Phytophthora than a control plant comprising a native type 2A serine/threonine protein phosphatase (PP2A) subunit A, the method comprising,
introducing a modification in the native PP2A subunit A to form a modified native PP2A subunit A, or knocking out the native PP2A subunit A in a plant, and
following the introducing, testing the plant for susceptibility to Phytophthora

9. The method of claim 8, wherein the modified native PP2A subunit A is at least 70, 75, 80, 85, 90, or 95% identical to one or more of SEQ ID NO: 1, 2, 3, 4, 5, 6, 7, 8, 9, 10, or 11.

10. The method of claim 8, wherein the plant comprises the modified native type 2A serine/threonine protein phosphatase (PP2A) subunit A.

11. The method of claim 10, wherein the modification is a point mutation compared to the native PP2A subunit A.

12. The method of claim 10, wherein the modification is a deletion or truncation compared to the native PP2A subunit A.

13. The method of claim 8, wherein the method comprises knocking out the native PP2A subunit A in the plant.
